(19) United States
(12) Patent Application Publication
(10) Pub. No.: US 2021/0395576 A1
(43) Pub. Date: Dec. 23, 2021

(54) PRESSURE SENSITIVE ADHESIVES FROM POLYMERIZATION OF ACETOACETATES AND ACRYLATES

(71) Applicant: Oregon State University, Corvallis, OR (US)

(72) Inventors: Kaichang Li, Corvallis, OR (US); Jian Huang, Corvallis, OR (US)

(73) Assignee: Oregon State University, Corvallis, OR (US)

(21) Appl. No.: 17/288,446

(22) PCT Filed: Oct. 30, 2019
(86) PCT No.: PCT/US2019/058869

§ 371 Date: Apr. 23, 2021

Related U.S. Application Data
(60) Provisional application No. 62/753,542, filed on Oct. 31, 2018.

(51) Int. Cl. C09J 7/38; C09J 7/25; A61L 15/24; A61L 15/58

(52) U.S. Cl. CPC C09J 7/385; C09J 7/255; A61L 15/24; C09J 2203/358; C09J 2301/302; C09J 2433/00; C09J 2467/006; A61L 15/58

(57) ABSTRACT

A pressure sensitive adhesive composition comprising a product made from:
(i) an acetoacetic ester;
(ii) a diacrylate;
(iii) a crosslinking agent that contains more than two acryloyl groups; and
(iv) a catalyst.

This application claims the benefit of U.S. Provisional Appl. No. 62/753,542, filed Oct. 31, 2018, which is incorporated herein by reference.

### BACKGROUND

Pressure-sensitive adhesive (PSA, also known as “self-adhesive” or “self-stick adhesive”) is a distinct category of adhesives which in dry form (solvent/water free) are aggressively and permanently tacky at room temperature. PSAs form a bond at room temperature with a variety of dissimilar surfaces when light pressure is applied. No solvent, heat or radiation is needed to activate the adhesive. They find wide applications in pressure-sensitive tapes, general purpose labels, post-it notes, postage stamps, and a wide variety of other products, e.g., packaging, automobile trim assembly, sound/vibration damping films, maternity and child care products such as diapers, and hospital and first aid products such as wound care dressings

### SUMMARY

One embodiment disclosed herein is a pressure sensitive adhesive composition comprising a product made from:

(i) an acetoacetic ester;

(ii) a diacrylate;

(iii) a crosslinking agent that contains more than two acryloyl groups; and

(iv) a catalyst.

Another embodiment disclosed herein is a method for making a pressure sensitive adhesive comprising combining:

(i) an acetoacetic ester;

(ii) a diacrylate;

(iii) a crosslinking agent that contains more than two acryloyl groups; and

(iv) a catalyst.

A further embodiment disclosed herein is a pressure sensitive adhesive construct comprising:

(A) a backing substrate; and

(B) a pressure sensitive adhesive composition disposed on the backing substrate, wherein the pressure sensitive adhesive composition includes a product made from:
(i) an acetoacetic ester;
(ii) a diacrylate;
(iii) a crosslinking agent that contains more than two acryloyl groups; and
(iv) a catalyst.

The foregoing will become more apparent from the following detailed description, which proceeds with reference to the accompanying figures.

### DETAILED DESCRIPTION

The term “pressure sensitive adhesive” as used herein refer to adhesives that possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as PSAs are polymers designed and formulated to exhibit the requisite viscoelastic properties resulting in a desired balance of tack, peel adhesion, and shear holding power.

Disclosed herein are pressure sensitive adhesives (PSAs) that are prepared from the polymerization of a mixture comprising, or consisting of, an acetoacetate, a diacrylate, a crosslinking agent containing more than two acryloyl groups, and a base catalyst. The polymerization is via Michael addition reaction. The acetoacetate is a Michael donor. The acryloyl groups in the diacrylate and the crosslinking agent are Michael acceptors. In certain embodiments, the adhesives have low peel strength, excellent adhesion to human skin, easy removal from the skin, and/or do not cause any reactions such as irritation to the skin.

The acetoacetate may be an acetoacetic ester. In certain embodiments, the acetoacetic ester is an acetoacetic alkyl ester, particularly an acetoacetic (C₁-C₄) alkyl ester. In certain embodiments, the acetoacetic ester has a molecular weight of 240 or less. Illustrative acetoacetic esters include methyl acetoacetate, ethyl acetoacetate, propyl acetoacetate, isopropyl acetoacetate, butyl acetoacetate, isobutyl acetoacetate, tent-butyl acetoacetate, 2-methoxyethyl acetoacetate, benzyl acetoacetate, or a mixture thereof.

In certain embodiments, the acetoacetate is present in an amount less than 25% by weight, based on the total dry weight of the composition mixture. In certain embodiments, the acetoacetate is present in an amount of 1% to 25% by weight, based on the total dry weight of the composition mixture.

The diacrylate may be any molecule that includes two acryloyl groups. Illustrative diacrylates include poly(propylene glycol) diacrylate, poly(1,3-propanediol) diacrylate, 1,6-hexanediol diacrylate, bisphenol A glycerolate(1 glycerol/phenol) diacrylate, poly(ethylene glycol) diacrylate, ethylene glycol diacrylate, an acrylated vegetable oil with two acryloyl groups per molecule, or a mixture thereof.

In certain embodiments, the diacrylate is present in an amount of 20% to 95% by weight, based on the total dry weight of the composition mixture.

The crosslinking agent contains more than two acryloyl groups. Illustrative crosslinking agents include trimethylolpropane triacrylate, pentaerythritol triacrylate, trimethylolpropane ethoxylate triacrylate, glycerol propoxylate (1PO/OH) triacrylate, trimethylolpropane propoxylate triacrylate, pentaerythritol tetraacrylate, di(trimethylolpropane) tetraacrylate, a (meth)acrylated vegetable oil with more than two acryloyl groups per molecule, or a mixture thereof.

In certain embodiments, the crosslinking agent is present in an amount of 1% to 70% by weight, based on the total dry weight of the composition mixture.

An illustrative (meth)acrylated vegetable oil is acrylated epoxidized soybean oil (AESO). Vegetable oils are triglycerides of glycerol and fatty acids extracted from plant materials. Typically, the fatty acids are long chain (C₁₂ to C₂₄ or even longer) materials with multiple double bonds per chain. The vegetable oil can be palm oil, olive oil, canola oil, corn oil, cottonseed oil, soybean oil, linseed oil, rapeseed oil, castor oil, coconut oil, palm kernel oil, rice bran oil, safflower oil, sesame oil, sunflower oil, or other polyunsaturated vegetable oils (both naturally existing and genetically modified), or mixtures thereof.

In compounds with activated C═C bonds acrylate groups can be readily introduced onto fatty chains via at least three methods: 1) direct reactions of acrylic acid with unsaturated vegetable oils in the presence of a strong acid catalyst result in acrylated vegetable oils (AVOs), 2) vegetable oil (e.g., soybean oil) can be readily epoxidized to form epoxidized soybean oil that can readily react with acrylic acid to form acrylated vegetable oils (AVOs) (e.g., acrylated epoxidized soybean oil (AESO)), and 3) the C═C bond in vegetable oils are first converted to a diol (—CH(OH)CH(OH)—) or mono-alcohol (—CH₂CH(OH)— or —CH₂CH(CH₂OH)—) that can further react with acrylic acid/acryloyl chloride/acrylic anhydride to form acrylated vegetable oils (AVOs). AVOs disclosed herein include all modified vegetable oils prepared in accordance with one of the previously described methods Methacrylic acid/methacryloyl chloride/methacrylic anhydride can be used to replace acrylic acid/acryloyl chloride/acrylic anhydride, respectively, in the previously described methods.

AVO is not a VOC or HAP. There are three long hydrocarbon chains in each AVO molecules and the C═C bonds of acrylate groups in AVO are located inside the long hydrocarbon chains.

The epoxidized vegetable oils (EVO) may be made from a vegetable oil by converting at least a portion of vegetable oil's double bonds into more reactive epoxy moieties. In particular embodiments, “EVO” generally refers to any derivative of vegetable oils whose double bonds are fully or partly epoxidized using any method, e.g. so called in situ performic acid process, which is the most widely applied process in industry. In certain embodiments, more than one EVO can be utilized in a single mixture if desired. EVOs generally have a functionality (including epoxy groups and possibly hydroxyl groups thereof) well above two. EVOs such as ESO and epoxidized linseed oil are also readily available from commercial suppliers such as Spectrum Chemical Mfg Corp, California, and Sigma-Aldrich Corp, Missouri.
The EVO may contain about 1 to about 9 epoxy groups (or even more) per triglyceride. It is preferred that the EVO contain functionality (epoxy number) of 2 to 7, more preferably 3 to 5. The epoxy functionality of EVO can be controlled by epoxidizing less than all of the double bonds of the starting vegetable oils.

Acrylated epoxidized vegetable oils (AEVO) may be made from the reaction of acrylic acid with EVO. For example, acrylated epoxidized soybean oil (AESO) is made from the reaction of acrylic acid with epoxidized soybean oil (ESO). Methacrylic acid can be used for replacement of acrylic acid in its reaction with EVO to generate methacrylated epoxidized vegetable oils (MEVO). The designation “(meth)acrylate” and similar designations are used as abbreviated notation for “acrylate or methacrylate”.

In the presence of an oxidant such as hydrogen peroxide and a catalyst such as a molybdenum, tungsten, or rhenium-based catalyst, the C═C bond in vegetable oils can be hydroxylated to form a vicinal diol (—CH(OH)CH(OH)—) that can further react with (meth)acrylic acid/(meth)acryloyl chloride/(meth)acrylic anhydride to form (meth)acrylated vegetable oils.

Vegetable oils can also react with formaldehyde in the presence of a catalyst to form hydroxymethylated vegetable oils that can further react with (meth)acrylic acid/(meth)acryloyl chloride/(meth)acrylic anhydride to form (meth)acrylated vegetable oils.

(Meth)acrylated vegetable oils can also be produced through direction reactions of (meth)acrylic acid with unsaturated vegetable oils in the presence of a strong acid.

Vegetable oils can react with acetonitrile in the presence of a strong acid such as sulfuric acid to introduce acrylamide groups onto the C=C bonds via the Ritter reaction.

Catalysts for Michael Addition between acetoacetates and acrylates include 1,8-diazabicyclo[5.4.0]undec-7-ene (DBU), 1,5-diazabicyclo-[4,3,0]-non-5-ene. 1,5,7-triazabicyclo[4.4.0]dec-5-ene (TBD), 1,1,3,3-tetramethylguanidine (TMG), or a mixture thereof.

In certain embodiments, the catalyst is present in an amount of 0.02% to 10% by weight, based on the total dry weight of the composition mixture.

The PSA compositions may also include additives and fillers. Fillers may either originally occur in the starting materials such as the acetoacetate, diacrylate or crosslinking agent, or be included on purpose. Additives such as tackifiers, colored pigments, opacifiers, processing oil plasticizers, solvents and other constituents t may be incorporated in the PSAs.

In certain embodiments, the molar ratio of acetoacetate/diacrylate/crosslinking agent may range from 1/0.1/0.7 to 1/0.98/0.01, based on the moles of each material. In certain embodiments, the molar ratio of acetoacetate/diacrylate/crosslinking agent is 1/0.9/0.07. In certain embodiments, the molar ratio of acetoacetate/diacrylate/crosslinking agent is 1/0.75/0.13.

In certain embodiments, the acetoacetate, diacrylate, and crosslinking agent are the only reactive components in the ingredient composition. As used herein, “reactive component” means that the component chemically reacts with another component in the ingredient composition.

The polymers of the compositions generally possess glass transition temperatures T_{g} at or below room temperature and have appropriate density of chemical and/or physical crosslinks, which render the polymers a balance between sufficient cohesive strength (“dry”) and good initial tack and adhesive power. For example, the T_{g} of the PSA compositions may be from 0 to −60° C., preferably from −20 to −60° C., more preferably from −30 to −60° C.

In certain embodiments, the pressure sensitive adhesive composition is biodegradable. For example, the composition will decompose when placed in a landfill or similar environment because of the degradable ester linkages present in the polymer structure.

In certain embodiments, the polymer product disclosed herein is the majority component of the pressure sensitive adhesive composition meaning the pressure sensitive adhesive composition includes at least about 50, particularly at least about 70, more particularly at least about 80, and most particularly at least about 90, weight percent of the polymer product based on the total weight of the pressure sensitive adhesive composition.

The components of the composition may be admixed in any combination, in any order. In certain embodiments, the components will be added simultaneously or in sequence to a container and admixed. In certain embodiments, two or more ingredients will be mixed together and stored as a mixture (herein called a “part”), to be combined with further ingredients at a later time to form the mixture. When some ingredients are mixed together to form a part, the remaining ingredients, even if stored in pure form, will also be called “parts” herein. In certain embodiments, the mixture is a two-part system. The term “two part” is used herein to mean that all the ingredients necessary for Michael addition to occur are contained in the admixture obtained by admixing the first part and the second part. In certain embodiments, the first part contains the acetoacetate, and the second part contains the diacrylate and the crosslinking agent.

In the practice of two-part, one or both of the first part and the second part contains at least one catalyst. The catalyst may be added at any point during the polymerization from the initial charge until the coating of the reaction mixtures onto a substrate. In certain embodiments, it is important that the catalyst be added when the catalyst can be homogeneously distributed.

In certain embodiments, one or more of the ingredients of the mixture are dissolved in a solvent or otherwise carried in a fluid medium (for example, as an emulsion or dispersion). If a solvent or other fluid medium is used with one or more ingredients, the solvents or other fluid media of the plural ingredients may be chosen independently of each other. In some embodiments, the mixture is substantially free of solvent. As defined herein, a material is “substantially free of solvent” if that material contains at least 75% “solids” by weight based on the total weight of that material. By “solids” is meant herein all materials that have a boiling point above 150° C. at 760 mmHg. In certain embodiments, the acetoacetic ester and the diacrylate are liquids at room temperature.

In some embodiments, the mixture is at least 80% solids, or at least 90% solids, or at least 95% solids, or at least 98% solids, by weight based on the total weight of the mixture.

Also contemplated are “low solids” embodiments, which are embodiments in which the mixture contains less than 75% solids by weight based on the weight of the mixture. In some low solids embodiments, the solids may be dissolved in a fluid medium or dispersed in a fluid medium or a combination thereof.

The mixture is formed by admixing the ingredients; the admixing may be performed by any means. In some embodiments, the ingredients are all liquids, and they may be admixed simply by placing the ingredients in a container and stirring. If any ingredient is a solid, it is contemplated that sufficient agitation will be provided to dissolve or suspend the solid in the mixture.

The mixture, when it is freshly mixed, should have a useful viscosity at 23° C. In some embodiments, the freshly-mixed functional mixture will be a liquid with viscosity of 1 cps or higher. In some embodiments the freshly-mixed mixture will be a liquid with viscosity of 500,000 cps or less.

In certain embodiments, the mixture is cured at 10° C. to 50° C. in 20 days or less.

In certain embodiments, the pot life of the admixture that includes all the ingredient is 10 days or less at 10° C. to 50° C. The pot life is the time from the formation of the admixture until the viscosity of the mixture rises so high that the admixture can no longer be applied to a substrate at 10° C. to 50° C.

According to particular embodiments, the disclosed PSAs may be used to manufacture many different types of PSA constructs or articles. Thus, various flexible backings and liners may be used, including films (transparent and non-transparent), plastics such as PET film, BOPP and PVC film or modified natural substances such as cellophane, cloths, papers, non-woven fibrous constructions, metal foils, metalized plastics foils, aligned filaments, etc. The adhesive layers can be covered with paper or films which contain an adhesive-repellent layer, e.g. a separating layer consisting of silicone, for the protection of the adhesive layers on the PSA backings. The back side of the PSA films, tapes or foils can be coated with an adhesive-repellent coating (e.g., silicone coating) for facilitating rolling off the PSA.

In certain embodiments, the PSA disclosed herein is useful for medical tapes and dressings because the PSA can provide adhesion while gently removing from the skin without causing trauma or stripping skin cells or hair.

In certain embodiments, the PSA disclosed herein is useful with absorbent articles such as diapers, training pants, sanitary napkins, incontinence garments, bandages, or skin treatment coverings.

In some embodiments, the adhesive article is suitable for application to skin. Therefore, the adhesive article can be a medical tape, bandage, or wound dressing. In some embodiments, the adhesive article can be an IV site dressing, a buccal patch, or a transdermal patch. In some embodiments, the adhesive articles according to the present disclosure may be adhered to the skin of humans and/or animals. The adhesive articles may include other materials such as polymeric materials, plastics, natural macromolecular materials (e.g., collagen, wood, cork, and leather), paper, films, foams, woven cloth and non-woven cloth, composites, and combinations of these materials.

In some embodiments, substrates included in the adhesive articles include a thermoplastic polyurethane, a thermoplastic polyester, or a combination thereof. The thermoplastic polyurethane or thermoplastic polyester may be selected to have a high moisture vapor rate. Such substrates are useful, for example, in absorbent articles. In some embodiments, the substrate includes a thermoplastic polyurethane. A variety of thermoplastic polyurethanes may be useful, including polyether-based polyurethanes and polyester-based polyurethanes. For example, a polyether or polyester with isocyanate-reactive terminal groups may be reacted with an aryl, alkyl, arylalkylenyl, or cycloalkyl diisocyanate to provide a useful polyurethane. Exemplary suitable polyurethanes include those commercially available, for example, from Lubrizol, Wickliffe, Ohio, under the trade designation “ESTANE” (e.g., “ESTANE 58237”, “ESTANE 58309”, or “ESTANE 58213”) and from Huntsman, The Woodlands, Tex., under the trade designation “IROGRAN” (e.g., “IROGRAN A 60 E 4902”). A variety of thermoplastic polyesters may also be useful. Exemplary suitable polyesters include those commercially available, for example, from Mitsubishi, Greer, S.C., under the trade designation “HOSTAPHAN 3SAB” polyester film and from E.I. du Pont de Nemours and Company, Wilmington, Del., under the trade designation “HYTREL”.

### EXAMPLE 1

Measurement of the peel strength was carried out on a stainless steel panel in accordance with ASTM D3330/D3330M-04 (Standard Test Method for Peel Adhesion of Pressure-Sensitive Tape). Briefly, tests were performed at 23±1° C. and 40±5% RH. The stainless steel panel was cleaned with ethanol and dried under a heat gun before each test. One end of a PSA specimen tape (24 mm by 150 mm) was applied onto the stainless steel panel and then pressed with a roller with a weight of 2040 g twice in the lengthwise direction. The other free end of the tape was attached to the grip on the load cell of a tensile tester. The distance from the grip to the surface of the stainless steel panel was about 25 mm. The tape was then peeled upwards at 180° angle at a speed of 5 mm/s; the force required to peel off the tape from the test panel was recorded as peel strength. Five specimens were tested for each PSA sample and the averaged value in N/in. was reported as the peel strength.

### EXAMPLE 2

The loop tack of the PSA tapes was measured on a stainless steel panel (Type 302) in accordance with Test Method A of ASTM D6195-03. A loop that was prepared from a PSA strip and with the PSA facing outwards was brought into contact with the test panel with a contact area of 25 by 25 mm without external pressure applied. The loop was then immediately pulled away from the test panel at a speed of 5 mm s_1; the maximum force required to remove the PSA loop from the test panel was recorded as the loop tack. For an aging test, samples of the PSA tapes were placed in an Isotemp 625D incubator (Thermo Fisher Scientific Inc., Waltham, Mass.) at 60° C. After one week, the sample tapes were taken out of the incubator and conditioned at 23±1° C. and 40±5% RH for 16 h. PSA specimens were then prepared from the aged sample tapes and measured for their peel strength and loop tack in accordance with the methods described above.

### EXAMPLE 3

Ethyl acetoacetate (EAA) (4.92 g), poly(propylene glycol) diacrylate (Mn=800) (PPGD) (26.64 g) and acrylated epoxidized soybean oil (AESO) (3.18 g) from Sigma Aldrich (2.7 acryloyl groups per one triglyceride) were mixed at room temperature to generate a homogenous mixture. Then 1,5-diazabicyclo-[4,3,0]-non-5-ene (DBN) (0.18 g) were added and mixed well. The resulted mixture was stored at RT for about 2 h and then was coated onto a PET film. The coating was immediately laminated with a siliconized release PET film at a pressure of 45 psi to give a PET film/reaction mixture/release paper laminate. The reaction mixture in the laminate was fully cured at room temperature for 24 h to give a PSA.

### EXAMPLE 4

EAA (0.82 g), PPGD (4.44 g) and trimethylolpropane triacrylate (TMPT) (0.16 g) were mixed at room temperature to generate a homogenous mixture. Then DBN (0.05 g) were added and mixed well. The resulted mixture was stored at RT for about 45 min and then was coated onto a PET film. The coating was immediately laminated with a siliconized release PET film at a pressure of 45 psi to give a PET film/reaction mixture/release paper laminate. The reaction mixture in the laminate was fully cured at room temperature for 24 h to give a PSA.

### EXAMPLE 5

tert-Butyl acetoacetate (1.0 g), PPGD (4.44 g) and TMPT (0.16 g) were mixed at room temperature to generate a homogenous mixture. Then DBN (0.05 g) were added and mixed well. The resulted mixture was stored at RT for about 1 h and then was coated onto a PET film. The coating was immediately laminated with a siliconized release PET film at a pressure of 45 psi to give a PET film/reaction mixture/release paper laminate. The reaction mixture in the laminate was fully cured at room temperature for 24 h to give a PSA.

TABLE 1
Properties of PSAs prepared by Michael addition

| Example# | PB(N/in) | PA(N/in) | TB(N/in².) | TA(N/in².) |
|---|---|---|---|---|
| 3 | 0.1 | 0.2 | 1.5 | 2.0 |
| 4 | 0.1 | 0.2 | 1.3 | 1.6 |
| 5 | 0.2 | 0.4 | 1.5 | 1.8 |

PB: peel strength before the aging test
PA: peel strength after the aging test
TB: tack strength before the aging test
TA: tack strength after the aging test

In view of the many possible embodiments to which the principles of the disclosed invention may be applied, it should be recognized that the illustrated embodiments are only preferred examples of the invention and should not be taken as limiting the scope of the invention.

1. A pressure sensitive adhesive composition comprising a product made from:
(i) an acetoacetic ester;
(ii) a diacrylate;
(iii) a crosslinking agent that contains more than two acryloyl groups; and
(iv) a catalyst.

2. The composition of claim 1, wherein the acetoacetic ester is an acetoacetic alkyl ester.

3. The composition of claim 1, wherein the acetoacetic ester is an acetoacetic (C₁-C₄) alkyl ester.

4. (canceled)

5. The composition of claim 1, wherein the acetoacetic ester is methyl acetoacetate, ethyl acetoacetate, propyl acetoacetate, isopropyl acetoacetate, butyl acetoacetate, isobutyl acetoacetate, tert-butyl acetoacetate, 2-methoxyethyl acetoacetate, benzyl acetoacetate, or a mixture thereof.

6. The composition of claim 1, wherein the acetoacetic ester is ethyl acetoacetate.

7. The composition of claim 1, wherein the diacrylate is poly(propylene glycol) diacrylate, poly(1,3-propanediol) diacrylate, 1,6-hexanediol diacrylate, bisphenol A glycerolate(1 glycerol/phenol) diacrylate, poly(ethylene glycol) diacrylate, ethylene glycol diacrylate, an acrylated vegetable oil with two acryloyl groups per molecule, or a mixture thereof.

8. The composition of claim 1, wherein the diacrylate is poly(propylene glycol) diacrylate, acrylated vegetable oil with two acryloyl groups per molecule, or a mixture thereof.

9. The composition of claim 1, wherein the crosslinking agent is trimethylolpropane triacrylate, pentaerythritol triacrylate, trimethylolpropane ethoxylate triacrylate, glycerol propoxylate (1PO/OH) triacrylate, trimethylolpropane propoxylate triacrylate, pentaerythritol tetraacrylate, di(trimethylolpropane) tetraacrylate, a (meth)acrylated vegetable oil with more than two acryloyl groups per molecule, or a mixture thereof.

10. (canceled)

11. The composition of claim 9, wherein the (meth)acrylated vegetable oil is acrylated epoxidized soybean oil.

12. The composition of claim 1, wherein the catalyst is 1,8-diazabicyclo[5.4.0]undec-7-ene (DBU), 1,5-diazabicyclo-[4,3,0]-non-5-ene (DBN), 1,5,7-triazabicyclo[4.4.0]dec-5-ene (TBD), 1,1,3,3-tetramethylguanidine (TMG), or a mixture thereof.

13. (canceled)

14. The composition of claim 1, wherein the molar ratio of acetoacetic ester/diacrylate/crosslinking agent ranges from 1/0.1/0.7 to 1/0.98/0.01.

15. The composition of claim 1, wherein the acetoacetic ester is present in an amount less than 25% by weight, based on the total dry weight of the composition mixture.

16. The composition of claim 1, wherein the product is a polymer.

17. A method for making a pressure sensitive adhesive comprising combining:
(i) an acetoacetic ester;
(ii) a diacrylate;
(iii) a crosslinking agent that contains more than two acryloyl groups; and
(iv) a catalyst.

18. The method of claim 17, wherein the acetoacetic ester is ethyl acetoacetate; the diacrylate is poly(propylene glycol) diacrylate, acrylated vegetable oil with two acryloyl groups per molecule, or a mixture thereof; the crosslinking agent is trimethylolpropane triacrylate, (meth)acrylated vegetable oil with more than two acryloyl groups per molecule, or a mixture thereof; and the catalyst is 1,5-diazabicyclo-[4,3,0]-non-5-ene (DBN).

19. The method of claim 17, wherein the method comprises mixing the acetoacetic ester, the diacrylate, the crosslinking agent, and the catalyst.

20. A pressure sensitive adhesive construct comprising:
(A) a backing substrate; and
(B) a pressure sensitive adhesive composition disposed on the backing substrate, wherein the pressure sensitive adhesive composition includes a product made from:
(i) an acetoacetic ester;
(ii) a diacrylate;
(iii) a crosslinking agent that contains more than two acryloyl groups; and
(iv) a catalyst.

21. The construct of claim 20, wherein the acetoacetic ester is an acetoacetic alkyl ester.

22. (canceled)

23. The construct of claim 20, wherein the acetoacetic ester is methyl acetoacetate, ethyl acetoacetate, propyl acetoacetate, isopropyl acetoacetate, butyl acetoacetate, isobutyl acetoacetate, tert-butyl acetoacetate, 2-methoxyethyl acetoacetate, benzyl acetoacetate, or a mixture thereof.

24. The composition of claim 20, wherein the acetoacetic ester is ethyl acetoacetate.

25. The construct of claim 20, wherein the diacrylate is poly(propylene glycol) diacrylate, poly(1,3-propanediol) diacrylate, 1,6-hexanediol diacrylate, bisphenol A glycerolate(1 glycerol/phenol) diacrylate, poly(ethylene glycol) diacrylate, ethylene glycol diacrylate, an acrylated vegetable oil with two acryloyl groups per molecule, or a mixture thereof.

26. The construct of claim 20, wherein the diacrylate is poly(propylene glycol) diacrylate, acrylated vegetable oil with two acryloyl groups per molecule, or a mixture thereof.

27. The construct of claim 20, wherein the crosslinking agent is trimethylolpropane triacrylate, pentaerythritol triacrylate, trimethylolpropane ethoxylate triacrylate, glycerol propoxylate (1PO/OH) triacrylate, trimethylolpropane propoxylate triacrylate, pentaerythritol tetraacrylate, di(trimethylolpropane) tetraacrylate, a (meth)acrylated vegetable oil with more than two acryloyl groups per molecule, or a mixture thereof.

28. (canceled)

29. The construct of claim 27, wherein the (meth)acrylated vegetable oil is acrylated epoxidized soybean oil.

30. The construct of claim 20, wherein the catalyst is 1,8-diazabicyclo[5.4.0]undec-7-ene (DBU), 1,5-diazabicyclo-[4,3,0]-non-5-ene (DBN), 1,5,7-triazabicyclo[4.4.0]dec-5-ene (TBD), 1,1,3,3-tetramethylguanidine (TMG), or a mixture thereof.

31. (canceled)

32. The construct of claim 20, wherein the molar ratio of acetoacetic ester/diacrylate/crosslinking agent ranges from 1/0.1/0.7 to 1/0.98/0.01.

33. The construct of claim 20, wherein the acetoacetic ester is present in an amount less than 25% by weight, based on the total dry weight of the composition mixture.

34. The construct of wherein the construct is a diaper, a training pant, a sanitary napkin, an incontinence garment, a bandage, a skin treatment covering, a medical tape, or a wound dressing.

35-41. (canceled)

42. A pressure sensitive adhesive-forming product comprising:
a first part that comprises at least one ingredient selected from the group consisting of
(i) an acetoacetic ester;
(ii) a diacrylate; and
(iii) a crosslinking agent that contains more than two acryloyl groups; and
a second part that comprises at least one ingredient selected from the group consisting of
(i) an acetoacetic ester;
(ii) a diacrylate; and
(iii) a crosslinking agent that contains more than two acryloyl groups,
provided that neither the first part nor the second part includes all three of the ingredients (i), (ii) and (iii).

43. The pressure sensitive adhesive-forming product of claim 42, wherein the first part includes the acetoacetic ester but not the diacrylate and the crosslinking agent, and the second part includes the diacrylate and the crosslinking agent but not the acetoacetic ester.

44. The pressure sensitive adhesive-forming product of claim 42, further comprising a catalyst included in the first part, or the second part, or separately from the first part and the second part.

45. A polymer made by reacting (i) an acetoacetic ester; (ii) a diacrylate; and (iii) a crosslinking agent that contains more than two acryloyl groups, in the presence of a catalyst.

46. A pressure sensitive adhesive composition comprising the polymer of claim 45.
